(19) United States
(12) Patent Application Publication
(10) Pub. No.: US 2026/0232976 A1
(43) Pub. Date: Aug. 13, 2026

(54) Medication Delivery Device

(71) Applicants: SUMIDA CORPORATION, Tokyo (JP); NATIONAL UNIVERSITY CORPORATION, IWATE UNIVERSITY, Iwate (JP)

(72) Inventors: Kenji TERAO, Miyagi (JP); Masaki SAITO, Miyagi (JP); Akifumi SAKO, Miyagi (JP); Yosuke ARAKAKI, Miyagi (JP); Masahiro DAIBO, Morioka (JP)

(21) Appl. No.: 19/632,617

(22) Filed: Mar. 30, 2026

Related U.S. Application Data
(63) Continuation of application No. PCT/JP2024/023968, filed on Jul. 2, 2024.

(30) Foreign Application Priority Data
Nov. 2, 2023 (JP) 2023-188701

(51) Int. Cl. A61M 37/00; H01F 7/20; H01F 27/28

(52) U.S. Cl. CPC A61M 37/00; H01F 7/20; H01F 27/28; A61M 2037/0007; A61M 2210/0693

(57) ABSTRACT

A vector potential coil device generates a vector potential. A power supply device drives the vector potential coil device. A bed allows the vector potential coil device to be arranged so that the vector potential is applied to a target part in a living body to which a medication is delivered. The power supply device causes the vector potential coil device to generate the vector potential so that the medication is delivered to the target part by electrophoresis due to an electric field formed by the vector potential.

### CROSS REFERENCE TO RELATED APPLICATIONS

This application is a continuation of PCT Application No. PCT/JP 2024/023968, filed on Jul. 2, 2024, which claims priority to Japanese Patent Application No. 2023-188701, filed on Nov. 2, 2023. The contents of both of the above applications are expressly incorporated herein by reference in their entirety.

### BACKGROUND

### Technical Field

The present invention relates to a medication (medicines, medical substances, or drugs) delivery (administration) device.

### RELATED ART

One medication administration device uses an electroporation to administer medications transdermally (for instance, please refer to Japanese Patent Publication Number 2009-213585). Another method of a medication delivery is iontophoresis administration. Iontophoresis administration is a technique in which a weak electric current is conducted onto the skin surface and a charged medication is administered noninvasively and transdermally by electrophoresis.

Although iontophoresis administration is noninvasive, it requires conducting the weak electric current onto the skin surface. Thus, the iontophoresis administration requires an electrode(s) to be in contact with the skin. As a result, it imposes a great burden on a patient.

### SUMMARY

The present invention has been made in view of the above issue. The present invention has an object that is to obtain a medication (medicines, medical substances, or drugs) delivery (administration) device that delivers and administers medications noninvasively and in a noncontact manner.

A medication delivery device according to the present invention includes a vector potential coil device that generates a vector potential, a power supply device that drives the vector potential coil device, and an arranging (positioning) means that arranges the vector potential coil device so that the vector potential is applied to a target part (target site) in a living body to which a medication is delivered. Further, the power supply device causes the vector potential coil device to generate the vector potential so that the medication is delivered to the target part by electrophoresis due to an electric field formed by the above-mentioned vector potential.

### EFFECTS OF THE INVENTION

According to the present invention, it is possible to obtain a medication delivery device that delivers medications noninvasively and in a noncontact manner.

### BRIEF DESCRIPTION OF THE DRAWINGS

FIG. 1 is a block diagram that shows a configuration of a medication delivery device according to an embodiment of the present invention.

FIG. 2 is a side view that shows a medication delivery device according to a first embodiment of the present invention.

FIG. 3 is a diagram that shows a configuration of a vector potential coil device 1 according to a second embodiment of the present invention.

FIG. 4 is a diagram that shows a configuration of a vector potential coil device 1 with respect to a medication delivery device according to a third embodiment of the present invention.

FIG. 5 is a front view that shows an example of a vector potential coil according to a fourth embodiment of the present invention.

FIG. 6 is a top view that shows an example of the vector potential coil according to the fourth embodiment of the present invention.

FIG. 7 is a side view that shows an example of the vector potential coil according to the fourth embodiment of the present invention.

FIG. 8 is a plan view that shows a vector potential coil device 1 with respect to a medication delivery device according to a fifth embodiment of the present invention.

### DETAILED DESCRIPTION

Embodiments of the present invention will be explained below with reference to the drawings.

### First Embodiment

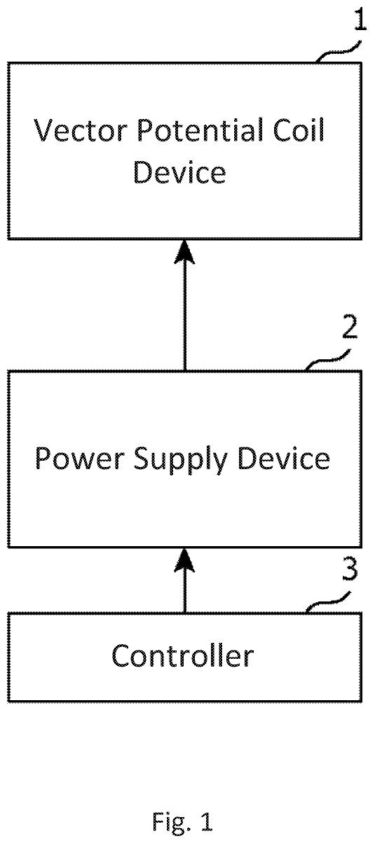

FIG. 1 is a block diagram that shows a configuration of a medication (medicines, medical substances, or drugs) delivery (administration) device according to an embodiment of the present invention. The medication delivery device shown in FIG. 1 is a device that generates a vector potential at a target part and delivers a medication to the target part by electrophoresis due to an electric field based on the vector potential. The medication delivery device has a vector potential coil device 1, a power supply device 2, and a controller 3 that controls the power supply device 2.

The vector potential coil device 1 has a vector potential coil (also referred to as a “VP coil” below). The VP coil is a solenoid coil extending along a coil axis of a specific shape and generates a vector potential therearound corresponding to a current that is conducted therein.

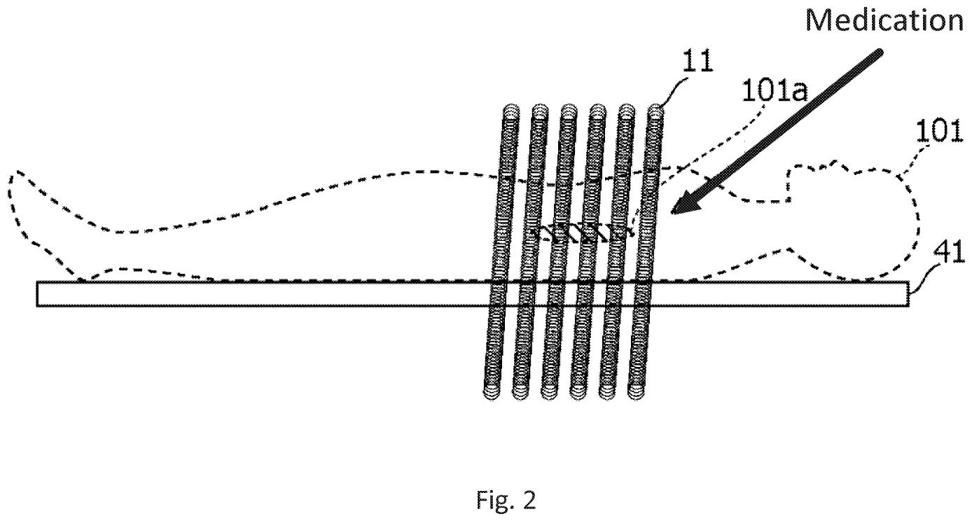

FIG. 2 is a side view that shows a medication delivery device according to a first embodiment of the present invention. As shown in FIG. 2, in the first embodiment, a VP coil 11 is a solenoid coil in which a helical (spiral) coil axis extends around an accommodating space for accommodating a living body 101. The solenoid coil is wound around the helical coil axis. Further, the outer shape of the VP coil 11 is substantially cylindrical. As a result, a vector potential that is substantially parallel to the central axis of the helical coil axis is generated.

The hollow portion of the substantially cylindrical VP coil 11 is the accommodating space for accommodating the living body (here, a human body) 101. A bed 41 for supporting the living body 101 (such as a patient) is arranged in the hollow portion. At least one of the bed 41 and the VP coil 11 is movable three-dimensionally relative to the other and is moved manually or electrically. As a result, as shown in FIG. 2, the VP coil 11 is arranged so that the vector potential is generated at or in a target part 101a.

Here, the target part 101a is, for instance, a specific subcutaneous tissue, a specific internal organ, a brain (specific part or site). In particular, for internal organs, there is no need to insert such as an electrode(s), and at the same time, for even deep inside the human body, an electrical stimulation can be applied directly using the vector potential.

That is, in the first embodiment, the bed 41 functions as an arranging (positioning) means that arranges the vector potential coil device 1 (the VP coil 11) so that the vector potential is applied to the target part 101a in the living body 101 to which a medication is delivered.

A vector potential generated by a current that is conducted through the VP coil 11 is weakened as the distance from the current increases. However, since the VP coil 11 (the coil axis thereof) is curved as mentioned above, an intensity of the vector potential becomes greater at an inner side (an inner direction) of the curvature (a curvature center in the case of a circular arc shape). Specifically, the vector potential being generated by the current at each position of the VP coil 11 overlaps at the inner side of the curvature.

Refer back to FIG. 1, the power supply device 2 generates a current based on power from, for instance, a commercial power supply or a battery (a primary battery or a secondary battery), and conducts that current (here, an alternating current (AC current) of a predetermined frequency) through the VP coil 11. Here, a waveform of the AC current may be a sine wave, a rectangular wave, a pulse wave, an impulse train (sequence), or a combination of these waves. Further, the AC current may be output steadily (constantly), or may be output intermittently in bursts that are repeatedly between output and stop.

Further, the controller 3 controls the power supply device 2 so as to cause the vector potential coil device 1 to generate a vector potential under a specified condition. As a result, the power supply device 2 causes the vector potential coil device 1 to generate the vector potential so that a medication is delivered to the target part 101a by electrophoresis due to the electric field formed by the vector potential.

Next, an operation of the medication delivery device according to the first embodiment will be explained.

The living body 101 is arranged on the bed 41, and the position of the living body 101 is adjusted as mentioned above by moving the bed 41. For instance, the position of the bed 41 is adjusted so that the target part 101a in the living body 101 on the bed 41 is arranged at the center of the above-mentioned accommodating space.

Further, the power supply device 2 causes the vector potential coil device 1 to generate a vector potential under a condition (such as a frequency, a waveform, and an intensity) specified by the controller 3. As a result, the vector potential of a sufficient intensity for the medication delivery is generated at or in the target part 101a.

Furthermore, when a medication is administered into the living body 101 by, for instance, an oral administration, a transdermal (dermal) administration, and an injection, and is guided to the vicinity of the target part 101a within the living body 101, the medication penetrates into the target part 101a by electrophoresis caused by the vector potential. Specifically, an electrical stimulation by the vector potential easily facilitates opening and closing of ion channels in a cell membrane, and the medication can penetrate into the target part 101a by electrophoresis.

As mentioned above, according to the first embodiment, the vector potential coil device 1 (the VP coil 11) generates the vector potential. The power supply device 2 drives the vector potential coil device 1. The bed 41 arranges the vector potential coil device 1 so that the vector potential is applied to the target part 101a in the living body 101 in which a medication is delivered. The power supply device 2 causes the vector potential coil device 1 to generate the vector potential so that the medication is delivered to the target part 101a by electrophoresis due to the electric field formed by the above-mentioned vector potential.

As a result, since the electrical stimulation is applied to the target part 101a noninvasively and in a noncontact manner by the vector potential, the medication delivery is carried out noninvasively and in a noncontact manner.

### Second Embodiment

In a medication delivery device according to a second embodiment, a solenoid coil in which a coil axis is not wound into a closed loop (in which the coil axis is less than one turn or linear) is used as a VP coil 11.

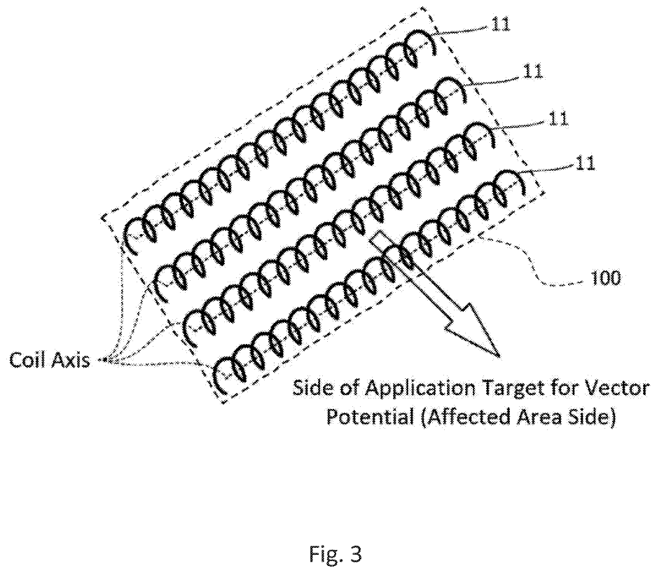

In addition, in the medication delivery device according to the second embodiment, the VP coil 11 is incorporated into and supported by a probe member 100 (see FIG. 3). Further, the probe member 100 is arranged at a position corresponding to the target part 101a in the living body 101. That is, in the second embodiment, the probe member 100 functions as the above-mentioned arranging (positioning) means.

FIG. 3 is a diagram that shows a configuration of a vector potential coil device 1 according to the second embodiment of the present invention. As shown in, for instance, FIG. 3, in the second embodiment, the vector potential coil device 1 has a plurality of VP coils 11. Each of the plurality of VP coils 11 in the second embodiment has a linear (straight) coil axis. Further, the plurality of VP coils 11 are a plurality of solenoid coils extending along the coil axes. The plurality of VP coils 11 are arranged along a linear arrangement direction. In other words, the outer shape of the vector potential coil device 1 is in a substantially flat plate shape. The power supply device 2 conducts a current through the plurality of VP coils 11. Further, the plurality of VP coils 11 may be electrically connected in series or in parallel to one another. Furthermore, a plurality of power supply devices 2 may conduct the current to the plurality of VP coils 11, respectively. In this case, the plurality of power supply device 2 respectively conduct an AC current through the plurality of VP coils 11 under the condition in which the AC currents being conducted through the plurality of VP coils 11 are synchronized. As mentioned above, by providing the plurality of VP coils 11, the intensity of the vector potential being applied to the application target becomes greater.

Note that the other configurations and operations of the medication delivery device according to the second embodiment are the same as those explained in any of the other embodiments. Therefore, the explanations of them will be omitted.

### Third Embodiment

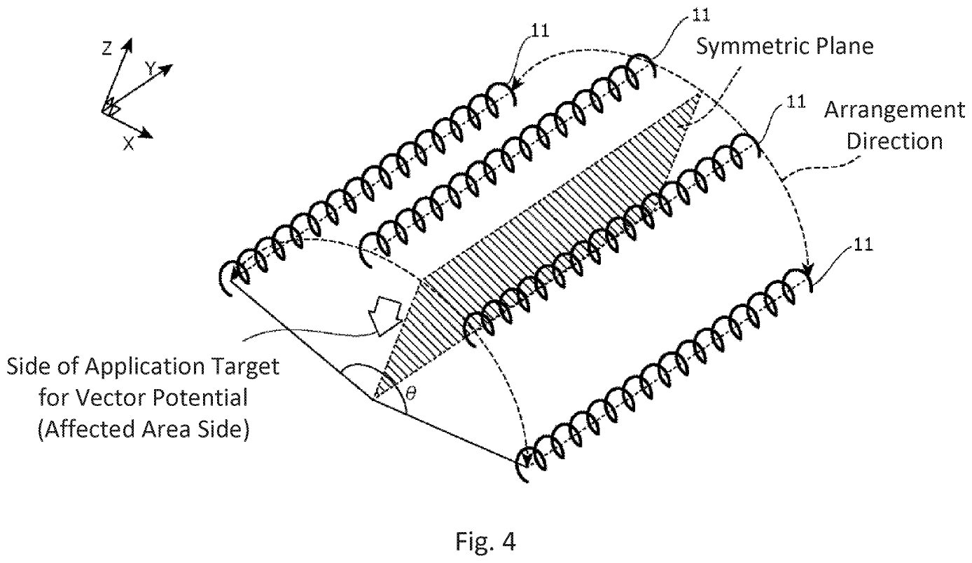

FIG. 4 is a diagram that shows a configuration of a vector potential coil device 1 with respect to a medication delivery device according to a third embodiment of the present invention. As shown in, for instance, FIG. 4, in the third embodiment, the vector potential coil device 1 has a plurality of VP coils 11. Each of the plurality of VP coils 11 in the third embodiment has a linear coil axis. Further, the plurality of VP coils 11 are a plurality of solenoid coils extending along the coil axes. The plurality of VP coils 11 are arranged along a curved (curvature) arrangement direction. The power supply device 2 conducts the current through the plurality of VP coils 11. Further, the plurality of VP coils 11 may be electrically connected in series or in parallel to one another. Here, the arrangement direction is a closed curve. Thus, the plurality of VP coils 11 are arranged along the circular-arc-shaped arrangement direction. In particular, the plurality of VP coils 11 are arranged within a range of a predetermined central angle θ (here, at an interval of an equal angle) with respect to a circle that includes the circular arc in the arrangement direction. Since the vector potentials of two of the VP coils 11 are canceled out at the intermediate position between the two VP coils 11, for instance, the central angle θ is set to be any angle less than 180 degrees.

For instance, in the same manner as the second embodiment, the plurality of VP coils 11 are incorporated into a probe member (for example, see FIG. 3). Further, the probe member is arranged so that the target part 101a in the living body 101 is located within a space in an inner side (direction) of the arranged plurality of VP coils 11.

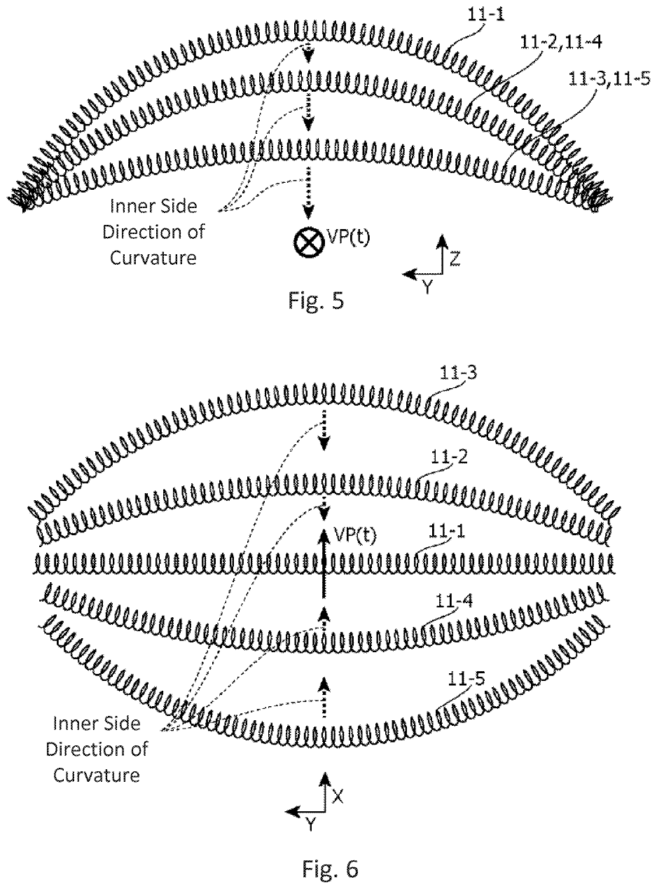

Note that as shown in, for instance, FIG. 4, when the plurality of VP coils 11 having the linear coil axes are arranged plane-symmetrically with respect to a predetermined symmetric plane (a plane perpendicular to the X-axis and parallel to the Z-axis and the Y-axis) along the curved arrangement direction, on an axis that passes through the center of the circle that includes the circular arc in the arrangement direction, and at the same time, is parallel to the coil axes, as a result of a vector synthesis (composition or combination) of the vector potentials that are generated by the plurality of VP coils 11, the vector potential is generated in a vertical (perpendicular) direction with respect to the symmetric plane (the X-axis direction in FIG. 4). Therefore, for instance, by combining the VP coil 11 having the curved coil axis shown in FIG. 5 with the plurality of VP coils 11 having the linear coil axes and being arranged plane-symmetrically with respect to the predetermined symmetric plane along the curved arrangement direction, it is possible to generate a vector potential in a desired direction within a two-dimensional plane of the X-axis and Y-axis.

Note that the other configurations and operations of the medication delivery device according to the third embodiment are the same as those explained in any of the other embodiments. Therefore, the explanations of them will be omitted.

### Fourth Embodiment

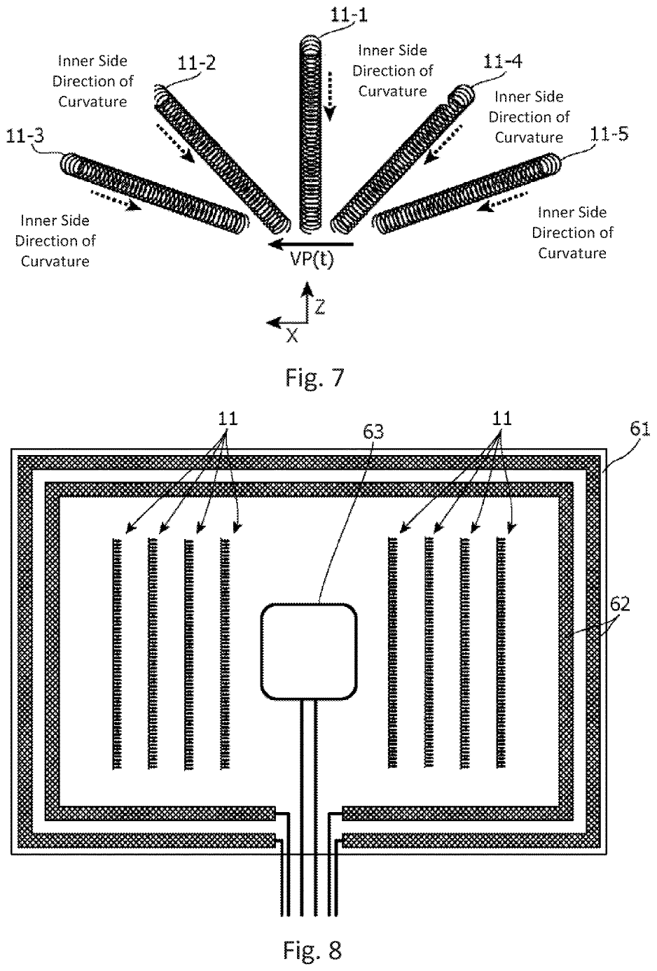

FIG. 5 is a front view that shows an example of a vector potential coil according to a fourth embodiment of the present invention. FIG. 6 is a top view that shows an example of the vector potential coil according to the fourth embodiment of the present invention. FIG. 7 is a side view that shows an example of the vector potential coil according to the fourth embodiment of the present invention.

The vector potential coil device 1 according to a fourth embodiment has a plurality of vector potential coils 11-1-11-5. As shown in, for instance, FIGS. 5-7, the plurality of vector potential coils 11-1-11-5 are respectively wound along (around) a curved coil axis, and are arranged so that the inner sides (directions) of the curvatures of the coil axes (in other words, planes that include the coil axes) cross mutually. For instance, as shown in FIG. 7, the plurality of vector potential coils 11-1-11-5 are arranged so that the planes that include the coil axes of the plurality of vector potential coils 11-1 to 11-5 are parallel to the Y-axis direction, and at the same time, the angle intervals of the inclination angles of these planes with respect to the X-axis direction are substantially the same. In addition, here, the inclination angle of the vector potential coil 11-1 is 90 degrees.

Note that, here, the vector potential coil device 1 has five of the vector potential coils 11-1-11-5. However, the vector potential coil device 1 may have the vector potential coils 11-1-11-M in the same manner as the configuration described above. The number M is either 2-4 coils or 6 or more coils.

For instance, the shape (such as the curvature) and the arrangement of the coil axes are determined so that the coil axes of the plurality of vector potential coils 11-1-11-5 are included in a single partial spherical surface (for instance, a semispherical surface). Further, the application target is arranged at the center of the spherical surface that includes that partial spherical surface (in other words, the center of curvatures of all of the coil axes). Further, the shape (such as the curvature) and the arrangement of the coil axes may be determined so that the coil axes of the plurality of vector potential coils 11-1-11-5 are included in a curved surface (a partial aspherical surface) other than a single partial spherical surface.

Further, the plurality of vector potential coils 11-1-11-5 respectively generate a vector potential according to the AC current in the same manner as the above-mentioned embodiments. The vector potentials by the plurality of vector potential coils 11-1-11-5 are synthesized (composited or combined) so that a vector potential VP(t) is obtained. Here, the power supply device 2 conducts the AC current through the plurality of vector potential coils 11-1-11-5 so that the amplitude of the synthesized (composited or combined) vector potential VP(t) becomes maximum (for instance, in the same phase mutually).

For instance, in the same manner as the second embodiment, the plurality of VP coils 11 are incorporated into a probe member (for example, see FIG. 3). Further, the probe member is arranged so that the target part 101a in the living body 101 is located within the space in the inner side (direction) of the arranged plurality of VP coils 11.

Note that the other configurations and operations of the medication delivery device according to the fourth embodiment are the same as those explained in any of the other embodiments. Therefore, the explanations of them will be omitted.

### Fifth Embodiment

FIG. 8 is a plan view that shows a vector potential coil device 1 with respect to a medication delivery device according to a fifth embodiment of the present invention. In the fifth embodiment, as shown in, for instance, FIG. 8, a plurality of VP coils 11 having the linear coil axes are arranged on a sheet-like member 61. The sheet-like member 61 may be a hard flat plate or a hard curved plate, or may be a flexible member such as a silicon sheet.

For instance, the sheet-like member 61 is arranged so that the target part 101a in the living body 101 is located within the space in the inner side (direction) of the arranged plurality of VP coils 11.

Further, in the fifth embodiment, the sheet-like member 61 is provided with a heater 62 (a resistor) for temperature control and an ultrasonic element 63 for ultrasonic control. In order to efficiently perform the opening and closing of the above-mentioned ion channel, when a vector potential is generated by the VP coils 11, the controller 3 also drives at least one of the heater 62 and the ultrasonic element 63 so as to adjust the temperature of the target part 101a and apply ultrasonic waves to the target part 101a. Furthermore, a light emitting device may be provided on the sheet-like member 61 to irradiate the target part 101a with light, thereby enabling the opening and closing of the ion channels to be carried out efficiently.

Note that the other configurations and operations of the medication delivery device according to the fifth embodiment are the same as those explained in any of the other embodiments. Therefore, the explanations of them will be omitted.

Note that various changes and modifications to the embodiments described above will be apparent to one having ordinally skill in the art. Such the changes and modifications may be made without departing from the spirit and scope of the subject matter and without diminishing the intended advantages. That is, it is intended that such the changes and modifications are included within the scope of the claims.

For instance, in the above-mentioned embodiments, after such as artificial insemination, in vitro fertilization (IVF), or microscopic insemination is performed, a vector potential may be applied to sperm by the above-mentioned medication delivery device to activate the sperm, thereby treating infertility. Since sperms contain voltage-dependent phosphatase molecules, sperms are expected to be activated by applying the vector potential.

### INDUSTRIAL APPLICABILITY

The present invention can be applicable to, for instance, a medication delivery device.

What is claimed is:

1. A medication delivery device comprising:
a vector potential coil device configured to generate a vector potential;
a power supply device configured to drive the vector potential coil device; and
an arranging structure configured to arrange the vector potential coil device so that the vector potential is applied to a target part in a living body to which a medication is delivered,
wherein the power supply device causes the vector potential coil device to generate the vector potential so that the medication is delivered to the target part by electrophoresis due to an electric field formed by the vector potential.

2. The medication delivery device according to claim 1,
wherein the vector potential coil device includes a solenoid coil in which a coil axis extends helically around an accommodation space for the living body, and
the arranging structure supports the living body within the accommodation space.

3. The medication delivery device according to claim 1,
wherein the vector potential coil device includes a plurality of solenoid coils arranged in a predetermined arrangement pattern, and
the arranging structure is a probe member or a sheet-like member that supports the plurality of solenoid coils.

4. The medication delivery device according to claim 1, wherein the target part is an internal organ or a brain.

5. The medication delivery device according to claim 2, wherein the target part is an internal organ or a brain.

6. The medication delivery device according to claim 3, wherein the target part is an internal organ or a brain.
